(19) United States
(12) Patent Application Publication
(10) Pub. No.: US 2026/0367010 A1
(43) Pub. Date: Nov. 19, 2026

(54) MIXTURE ADAPTED FOR MONITORING PRESENCE OF HYDROPEROXIDES IN A NON-AQUEOUS ENVIRONMENT AND RELATED METHODS

(71) Applicant: University of Kentucky Research Foundation, Lexington, KY (US)

(72) Inventor: William L. Boatright, Lexington, KY (US)

(21) Appl. No.: 19/674,920

(22) Filed: May 12, 2026

Related U.S. Application Data
(60) Provisional application No. 63/807,161, filed on May 16, 2025.

(51) Int. Cl. G01N 21/78; G01N 33/02; G01N 33/15

(52) U.S. Cl. CPC G01N 21/78; G01N 33/02; G01N 33/15

(57) ABSTRACT

A method of monitoring for the presence of hydroperoxides in a non-aqueous sample, includes steps of (a) contacting the non-aqueous sample with a mixture of a structured metal-phosphate complex that exhibits peroxidase-like activity and a colorimetric reagent wherein the structured metal-phosphate complex accepts an electron from the colorimetric reagent resulting in the colorimetric agent forming a detectable color and (b) measuring a color intensity produced by the colorimetric reagent.

### RELATED APPLICATION

This application claims priority to U.S. Provisional Patent Application Ser. No. 63/807,161, filed on May 16, 2025, hereby incorporated by reference.

### TECHNICAL FIELD

This document relates generally to the field of metal-phosphate semiconductors and, more particularly, to metal-phosphate semiconductors in combination with colorimetric reagents useful to detect and quantify the level of hydroperoxide in non-polar environments.

### BACKGROUND

“Reactive oxygen substances” or “ROS” means an active chemical species containing oxygen. Examples include hydroperoxides, peroxides, superoxide radical anion, hydroxyl radical and singlet oxygen. Free radicals are atoms or molecules with unpaired electrons. These radicals are highly reactive and can catalyze a variety of degradative reactions.

Current techniques for detecting hydroperoxides include fluorescent or spectrophotometric techniques (e.g., the Amplex Red and Leucocrystal Violet techniques); however, both of these techniques require an extensive incubation time for the development of the reaction product that is ultimately detected. There are a number of commercially available kits that can measure hydroperoxides and that are based on chromogen formation from ferric iron-xylenol orange complex or ferric thiocyanate. These kits exhibit an array of different problems including the use of toxic solvents/reagents, a lack of sensitivity and require expensive supportive instrumentation. Such problems are overcome with the method and mixture of compounds disclosed in this document.

Peroxide value (PV) as milliequivalents per kilogram is the most widely used analytical technique for measuring the degree of oxidation (as hydroperoxide content) in edible fats and oils, and peroxide number as milligrams per kilogram in petrochemicals. This titration technique was first developed for non-food applications in the 1880s. The term “peroxide value” was used in publications from the 1920s based on the observations that the development of rancidity was associated with the uptake of oxygen by edible oils. Lea published an iodometric method for the determination of peroxides in edible fats and oils by heating fat with powdered potassium iodide in a mixture of chloroform-acetic acid. This mixture was then titrated with sodium thiosulfate solution to measure the liberated iodine. The next year, Wheeler presented modifications to this iodometric titration method.

Official methods for measuring PV in edible oils, or peroxide number in petrochemicals, are established by the American Oil Chemists Society (AOCS) Official Method Cd 8b-90, International Union of Pure and Applied Chemistry (IUPAC) 2.501, International Organization for Standardization (ISO) 3960:2017, U.S. Pharmacopeial Convention methods (USP) 401 or American Society for Testing and Materials (ASTM) Method D3703. In 1949, an official method, based on the techniques of Lea and Wheeler, was adopted by the AOCS, and with the exception of the replacement of chloroform with isooctane in the 1990s, it has remained essentially the same.

Data presented in this document is the first to demonstrate the use of metal-phosphate complexes with peroxidase-like activity in combination with electron donor colorimetric reagents to develop a color response from reaction with hydroperoxides in non-polar environments such as edible fats and oils, renewable biofuels and petrochemicals. Furthermore, these color intensities can be used to quantify the level of hydroperoxides. These compounds exhibit concentration-dependent color intensities when reacted with standard solutions of cumene-hydroperoxide.

### SUMMARY

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, “substantially” is meant to permit deviations from the descriptive term that don't negatively impact the intended purpose. Descriptive terms are implicitly understood to be modified by the word substantially, even if the term is not explicitly modified by the word substantially.

As used herein, the singular forms “a,” “an” and “the” include plural referents unless the context clearly dictates otherwise.

As used herein, “about,” “approximate,” “at or about,” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated+10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “optional” or “optionally” means that the subsequently described event or circumstance can or cannot occur, and that the description includes instances where said event or circumstance occurs and instances where it does not.

In accordance with the purposes and benefits set forth herein, a new and improved method is provided for monitoring for the presence of hydroperoxides in a non-aqueous sample. That method comprises, consists of or consists essentially of: (a) contacting the non-aqueous sample with a mixture of a structured metal-phosphate complex that exhibits peroxidase-like activity and a colorimetric reagent wherein the structured metal-phosphate complex accepts an electron from the colorimetric reagent resulting in the colorimetric agent forming a detectable color and (b) measuring a color intensity produced by the colorimetric reagent.

The method may further include using a colorimetric reagent selected from a group consisting of 3, 3′,5,5′-tetramethylbenzidine (TMB), o-phenylenediamine (OPD), 2,2′-azinobis-(3-ethylbenzothiazoline-6-sulfonic acid (ABTS), olanzapine, 2,2′-dithiodipyridine (2-PDS), resazurin (AlamarBlue), bromothymol blue, zincon and mixtures thereof. The method may further include using a structured metal-phosphate complex selected from a group consisting of (a) a primary semiconductor material without a dopant prepared on a phytate scaffold, (b) a primary semiconductor material with a dopant prepared on a phytate scaffold, (c) a primary semiconductor material without a dopant prepared on a hexametaphosphate scaffold, (d) a primary semiconductor material with a dopant prepared on a hexametaphosphate scaffold, and combinations thereof.

In at least some embodiments, the method includes using a metal-phosphate complex (MPC) as the structured metal-phosphate complex exhibiting peroxidase-like activity. In some embodiments, the method includes using one or more scaffold materials to construct the MPC including phytic acid, a salt of phytic acid, hexametaphosphate, a hexametaphosphate salt and combinations thereof. Still further, the method may include using at least one metal component in the MPC. The at least one metal component may comprise manganese, zinc, calcium, manganese and calcium, or manganese and zinc.

In at least some embodiments, the measuring of the color development of the structured metal-phosphate complex and the colorimetric reagent is accomplished by using a reflectance colorimeter. In some embodiments, the measuring of the color development resulting from the structured metal-phosphate complex and colorimetric reagent is accomplished by making a visual determination with a color chart.

The method may include monitoring for the presence of the hydroperoxides in a non-polar environment. In some embodiments, the method includes monitoring for the presence of the hydroperoxides in food. In other embodiments, the method includes monitoring for the presence of the hydroperoxides in a pharmaceutical. In still other embodiments, the method includes monitoring for the presence of the hydroperoxides in a biological material. In yet another embodiment, the method includes monitoring for the presence of the hydroperoxides in a petrochemical material or dielectric cooling fluid.

The method may also include using the structured metal-phosphate complex with a phosphate scaffold provided at 3.0-80.0 percent by weight and manganese provided at 0.1-99.0 percent by weight. In some embodiments, the method includes providing manganese at about 30 percent by weight.

The method may include using a structured metal-phosphate complex with peroxidase-like activity having a phosphate scaffold derived from a blend of sodium hexametaphosphate and sodium tripolyphosphate or phytic acid.

In some embodiments, the method includes using a structured metal-phosphate complex with peroxidase-like activity having a phosphate scaffold derived from rice bran plasma ash.

In accordance with an additional aspect, a new and improved mixture is adapted for monitoring the presence of hydroperoxides in a non-aqueous environment. The mixture comprises, consists of or consists essentially of a structured metal-phosphate complex with peroxidase-like activity and a colorimetric reagent wherein the structured metal-phosphate complex with peroxidase-like activity accepts an electron from the colorimetric reagent resulting in the colorimetric agent forming a detectable color.

The colorimetric reagent may be selected from a group consisting of 3, 3′,5,5′-tetramethylbenzidine (TMB), o-phenylenediamine (OPD), 2,2′-azinobis-(3-ethylbenzothiazoline-6-sulfonic acid (ABTS), olanzapine, 2,2′-dithiodipyridine (2-PDS), resazurin (AlamarBlue), bromothymol blue, zincon and mixtures thereof.

The structured metal-phosphate complex with peroxidase-like activity may be selected from a group consisting of (a) a primary semiconductor material without a dopant prepared on a phytate scaffold, (b) a primary semiconductor material with a dopant prepared on a phytate scaffold, (c) a primary semiconductor material without a dopant prepared on a hexametaphosphate scaffold, (d) a primary semiconductor material with a dopant prepared on a hexametaphosphate scaffold, (e) and combinations thereof.

In some embodiments, the structured metal-phosphate complex is a metal-phosphate complex (MPC) that exhibits peroxidase-like activity. In some embodiments, the MPC includes phytic acid, a salt of phytic acid, hexametaphosphate, a hexametaphosphate salt and combinations thereof. In some embodiments, the MPC includes at least one metal component. The metal component may comprise: (a) manganese, (b) zinc, (c) calcium, (d) manganese and calcium, or (e) manganese and zinc.

In the following description, there are shown and described several embodiments of the method of monitoring for the presence of hydroperoxides in a non-aqueous sample and a mixture for doing the same. As it should be realized, the method and mixture are capable of other, different embodiments and their several details are capable of modification is various, obvious aspects all without departing from the apparatus and method as set forth and described in the following claims. Accordingly, the drawings and descriptions should be regarded as illustrative in nature and not as restrictive.

### BRIEF DESCRIPTION OF THE DRAWING FIGURE

FIG. 1 is a plot of International Commission on Illumination (CIE) Lab “a-values” vs peroxide values and related table for Example 2.

### DETAILED DESCRIPTION

As briefly outlined above, a method is provided for monitoring for a presence of hydroperoxides in a non-aqueous sample. That sample may comprise, for example, any non-aqueous and/or non-polar environment where the presence of hyperoxides are of interest. Such environments include, but are not necessarily limited to a food environment, a pharmaceutical environment, a biological environment and a petrochemical environment.

Food environments include, but are not necessarily limited to edible fats and oils.

Pharmaceutical environments include, but are not necessarily limited to glycerol and surfactants.

Biological environments include, but are not necessarily limited to phospholipids and other liquid classes.

Petrochemical environments include, but are not necessarily limited to kerosene, jet fuel, parrafins and naphthalene.

A composition or mixture of compounds adapted for monitoring for the presence of hydroperoxides in a non-aqueous environment is used in the method. The composition or mixture of compounds comprises (a) a structured metal-phosphate complex with peroxidase-like activity and (b) a colorimetric reagent wherein the structured metal-phosphate complex with peroxidase-like activity accepts an electron from the colorimetric reagent resulting in the colorimetric agent forming a detectable color. For purposes of this document the term “peroxidase-like activity” refers to the ability of a material to catalyze the oxidation of a peroxide (or hydroperoxide), with the simultaneous oxidizing of another molecule.

Examples of a structured metal-phosphate complex with peroxidase-like activity include, but are not necessarily limited to, luminophores such as a primary semiconductor material without a dopant prepared on a phytate scaffold, a primary semiconductor material with a dopant prepared on a phytate scaffold, a primary semiconductor material without a dopant prepared on a hexametaphosphate scaffold, a primary semiconductor material with a dopant prepared on a hexametaphosphate scaffold, and combinations thereof. For some applications, the structured metal-phosphate complex is a metal-phosphate complex (MPC) that exhibits peroxidase-like activity. Such an MPC may include phytic acid, a salt of phytic acid, hexametaphosphate, a hexametaphosphate salt and combinations thereof. Still further, the MPC includes at least one metal component such as (a) manganese, (b) zinc, (c) calcium, (d) manganese and calcium or (e) manganese and zinc.

In some useful embodiments, the structured metal-phosphate complex with a phosphate scaffold is provided at 3.0-80.0, 5.0-75.0, 10.0-70.0, 15.0-65.0, 20.0-60.0, 30.0-50.0, 35.0-45.0, about 30.0, or about 40.0 percent by weight and metal component is provided at 0.1-99.0, 5.0-95.0, 10.0-90.0, 15.0-85.0, 20.0-80.0, 25.0-75.0, 30.0-70.0, 35.0-65.0, 40.0-60.0, about 25, about 30, about 35, or about 40 percent by weight. For some applications, manganese is provided at about 30 percent by weight. The phosphate scaffold may be derived from a blend of sodium hexametaphosphate and sodium tripolyphosphate or phytic acid. Alternatively, the phosphate scaffold may be derived from rice bran plasma ash.

The colorimetric reagent of the composition or mixture of compounds may be selected from a group including 3, 3′,5,5′-tetramethylbenzidine (TMB), o-phenylenediamine (OPD), 2,2′-azinobis-(3-ethylbenzothiazoline-6-sulfonic acid (ABTS), olanzapine, 2,2′-dithiodipyridine (2-PDS), resazurin (AlamarBlue), bromothymol blue, zincon and mixtures thereof. This list of materials is to be considered representative and not restrictive.

The method may be broadly described as including the contacting of the non-aqueous sample with a mixture of the structured metal-phosphate complex that exhibits peroxidase-like activity and a colorimetric reagent of the composition or mixture of compounds previously described. In the presence of peroxidase or hydroperoxide, the structured metal-phosphate complex accepts an electron from the colorimetric reagent resulting in the colorimetric agent forming a detectable color. The method also includes the measuring of the color intensity produced by the colorimetric reagent. This may be accomplished by using a reflectance colorimeter or a UV-Visible Spectrophotometer. Alternatively, this may be accomplished by making a visual determination with a color chart.

The method may be used to monitor various environments for the presence of the hydroperoxides. Those environments include, but are not limited to (a) non-polar environments, food environments, (c) pharmaceutical environments, (d) biological material environments, (e) petrochemical environments, and (f) dielectric cooling fluid environments.

The following Experimental Section provides additional details relating to the composition or mixture of compounds and the method for detecting and quantifying the level of hydroperoxide in non-polar or non-aqueous environments.

### Experimental Section

### Application of Method

A method and mixture of a structured metal-phosphate complex with peroxidase-like activity and a colorimetric reagent material are provided to visualize, detect and even quantify oxidizing chemical species such as one or more reactive oxygen species (ROS) or combinations thereof. This is done via the development of a color that can be evaluated either visually or with a reflectance colorimeter. The phytic acid-based reagents with peroxidase like activity are set forth and described in U.S. Pat. Nos. 10,794,830, 10,788,426, which are fully incorporated herein by reference. The hexametaphosphate-based reagent is described in U.S. Pat. No. 12,043,779, which is also fully incorporated herein by reference. Both of these materials can be combined with colorimetric reagents to quantify hydroperoxides. Generally, the phytic acid-based material tends to generate a much stronger color for a given level of hydroperoxide. The hexametaphosphate-based material tends to provide enhanced light emission intensity.

As the hydroperoxides in petrochemicals are reduced by the peroxidase-like activity of the metal phosphate semiconductor reagent, the electron donor color reagent is converted from a colorless material into a colored end product. Overall this assay can be referred to as a Color Test. Such a Color Test may be achieved with a metal phosphate semiconductor reagent prepared using phytic acid/phytate coordinated manganese.

A similar series of color developments can be achieved with cumene-hydroperoxides in squalane to more closely match the viscosity of edible oils. All petrochemical systems tested generated similar color intensities for a given peroxide number, and all edible oils/squalane systems tested generate similar color intensities for a given peroxide value. Because of the much lower viscosities of petrochemicals the overall reaction proceeds more rapidly. Petrochemical Peroxide Number (PN) is reported as mg/Kg of hydroperoxide in fuel, or parts per million, while the Peroxide Value (PV) for edible oils is reported as milliequivalents/Kg of hydroperoxide in oils (meq/Kg). The milliequivalent weight of “total oxygen” in a hydroperoxide (whether petrochemical or fatty acid) is 16 mg. Thus, a peroxide value of 1 meq/Kg corresponds to a peroxide number of 16 mg/Kg.

For the edible oil standard color chart, and standard comparison, a dilution series of a standard hydroperoxide (e.g., cumene hydroperoxide) is diluted in squalane or silicone oil because of the similar viscosity. For the corresponding standard color chart for renewable diesel and petrochemicals the dilution is conducted with isooctane or hexane. The samples are developed with 100 μL of sample (or about 2 drops).

### Example 1 for Preparing Hexametaphosphate Scaffold

Briefly, mix 100 mLs water, 7 grams of sodium hexametaphosphate Technical grade (ca., 70%) and 5 grams sodium tripolyphosphate. Prepare premix by combining 39 mLs water with 34 mmoles calcium acetate and 8 mmoles manganese perchlorate. Add premix to sodium hexametaphosphate mixture and stir for 30 min. Filter slurry through filter paper (e.g., Whatman no. 42). Rinse the filtered material with 100 mLs water and then rinse with 50 mLs of methanol. Dry at 50 to 60 degrees C for 20 min, and store over silica gel desiccant overnight. Grind, sieve (e.g., less than or equal to 40 mesh) and store in an argon flushed desiccator over molecular sieve (e.g., 3A) for 48 hours before use.

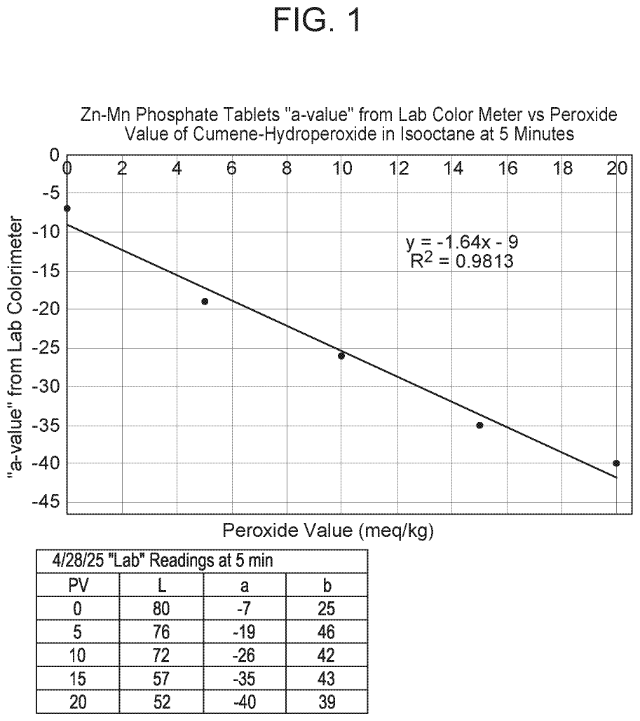

The “CIELab” color values, as defined by the International Commission on Illumination (CIE), using the same colorimeter and the same cumene hydroperoxide standards in isooctane (shown in FIG. 1; Example 2) are:

“CIELab” a-value Readings at 5 min

| PV | L | a | b |
|---|---|---|---|
| 0 | 80 | −7 | 25 |
| 5 | 76 | −19 | 46 |
| 10 | 72 | −26 | 42 |
| 15 | 57 | −35 | 43 |
| 20 | 52 | −40 | 39 |

The linear regression trend line and r-square values using the “a values” are: y=−1.64x-9 and r-square=0.9813.

### Example 2 for Preparing Phytic Acid Scaffold

Dissolve 12.5 grams phytic acid in 100 grams water and adjust pH to 8.0, followed by the addition of a 39 mL premix containing 34 mmoles zinc acetate dihydrate and 25 mmoles of manganese chloride. The mixture was held at 25±3 °C under agitation for 30 min. The slurry is then filtered through paper (e.g., Whatman no. 42). Wash with 100 mLs water followed by 50 mLs methanol. The filtered material is dried at 50 to 60 degrees C for 20 min, and stored in an argon flushed desiccator over silica gel desiccant overnight. Grind, sieve (e.g., less than or equal to 40 mesh) and store in an argon flushed desiccator over molecular sieve (e.g., 3A) for 48 hours before use.

### Details from Example 2 (Phytic Acid with Zinc-Manganese Luminophore with Peroxidase-Like Activity)

The MPC reagent and TMB (3, 3′,5,5′-tetramethylbenzidine) reagents were dry-mixed in a vortex mixer for 5 minutes. The 60 mg tablets of just these two reagents were pressed using a LFA Hand-Vice tablet press. Peroxide value series of cumene-hydroperoxide in isooctane was prepared using a serial dilution of a stock mixture. One tablet was placed in each of five wells on a 24-well cell-culture plate and 400 μL of the zero, 5, 10, 15, and 20 peroxide value (meq/Kg) cumene hydroperoxide solutions were added to each tablet. After 5 minutes of incubation, images were obtained, and the CIELAB color values (International Commission on Illumination) were obtained using a “Colorimeter Camera Color Meter” application (Contechity AB) on an Apple 13 mini iphone. The “a-values” were plotted vs peroxide values. See FIG. 1. Also, to obtain a rapid estimation of the peroxide values, tablet colors can be compared to a simple color chart.

Although the method and mixture of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A method of monitoring for a presence of hydroperoxides in a non-aqueous sample, comprising:
contacting the non-aqueous sample with a mixture of a structured metal-phosphate complex that exhibits peroxidase-like activity and a colorimetric reagent wherein the structured metal-phosphate complex accepts an electron from the colorimetric reagent resulting in the colorimetric agent forming a detectable color; and
measuring a color intensity produced by the colorimetric reagent.

2. The method of claim 1, further including using a colorimetric reagent selected from a group consisting of 3, 3′,5,5′-tetramethylbenzidine (TMB), o-phenylenediamine (OPD), 2,2′-azinobis-(3-ethylbenzothiazoline-6-sulfonic acid (ABTS), olanzapine, 2,2′-dithiodipyridine (2-PDS), resazurin (AlamarBlue), bromothymol blue, zincon and mixtures thereof.

3. The method of claim 1, including using a structured metal-phosphate complex selected from a group consisting of (a) a primary semiconductor material without a dopant prepared on a phytate scaffold, (b) a primary semiconductor material with a dopant prepared on a phytate scaffold, (c) a primary semiconductor material without a dopant prepared on a hexametaphosphate scaffold, (d) a primary semiconductor material with a dopant prepared on a hexametaphosphate scaffold, and combinations thereof.

4. The method of claim 1, including using a metal-phosphate complex (MPC) as the structured metal-phosphate complex exhibiting peroxidase-like activity.

5. The method of claim 4, including using one or more scaffold materials to construct the MPC including phytic acid, a salt of phytic acid, hexametaphosphate, a hexametaphosphate salt and combinations thereof.

6. The method of claim 4, including using at least one metal component in the MPC.

7. The method of claim 6, including using manganese, zinc, calcium, manganese and calcium, or manganese and zinc as the at least one metal component.

8. The method of claim 1, wherein the measuring of color development of the structured metal-phosphate complex and the colorimetric reagent is accomplished by using a reflectance colorimeter or a UV-Visible Spectrophotometer.

9. The method of claim 1, wherein the measuring of color development resulting from the structured metal-phosphate complex and colorimetric reagent is accomplished by making a visual determination with a color chart.

10. The method of claim 1, including monitoring presence of the hydroperoxides in a non-polar environment.

11. The method of claim 1, including monitoring presence of the hydroperoxides in food.

12. The method of claim 1, including monitoring presence of the hydroperoxides in a pharmaceutical.

13. The method of claim 1, including monitoring presence of the hydroperoxides in a biological material.

14. The method of claim 1, including monitoring presence of the hydroperoxides in a petrochemical material or dielectric cooling fluid.

15. The method of claim 1, including using the structured metal-phosphate complex with a phosphate scaffold provided at 3.0-80.0 percent by weight and manganese provided at 0.1-99.0 percent by weight.

16. The method of claim 15, including providing manganese at about 30 percent.

17. The method of claim 1, including using a structured metal-phosphate complex with peroxidase-like activity having a phosphate scaffold derived from a blend of sodium hexametaphosphate and sodium tripolyphosphate or phytic acid.

18. The method of claim 1, including using a structured metal-phosphate complex with peroxidase-like activity having a phosphate scaffold derived from rice bran plasma ash.

19. A mixture adapted for monitoring presence of hydroperoxides in a non-aqueous environment, comprising:
a structured metal-phosphate complex with peroxidase-like activity and a colorimetric reagent wherein the structured metal-phosphate complex with peroxidase-like activity accepts an electron from the colorimetric reagent resulting in the colorimetric agent forming a detectable color.

20. The mixture of claim 19 wherein (a) the colorimetric reagent is selected from a group consisting of 3, 3′,5,5′-tetramethylbenzidine (TMB), o-phenylenediamine (OPD), 2,2′-azinobis-(3-ethylbenzothiazoline-6-sulfonic acid (ABTS), olanzapine, 2,2′-dithiodipyridine (2-PDS), resazurin (AlamarBlue), bromothymol blue, zincon and mixtures thereof and (b) the structured metal-phosphate complex with peroxidase-like activity is selected from a group consisting of (i) a primary semiconductor material without a dopant prepared on a phytate scaffold, (ii) a primary semiconductor material with a dopant prepared on a phytate scaffold, (iii) a primary semiconductor material without a dopant prepared on a hexametaphosphate scaffold, (iv) a primary semiconductor material with a dopant prepared on a hexametaphosphate scaffold, (v) and combinations thereof.
